(19) United States
(12) Patent Application Publication
(10) Pub. No.: US 2024/0241099 A1
(43) Pub. Date: Jul. 18, 2024

(54) METHOD FOR IDENTIFYING PI3 KINASE-ALPHA INHIBITORS

(71) Applicant: UNIVERSITÄT BASEL, Basel (CH)

(72) Inventors: Chiara BORSARI, Basel (CH); Matthias WYMANN, Bern (CH)

(73) Assignee: UNIVERSITÄT BASEL, Basel (CH)

(21) Appl. No.: 18/554,638

(22) PCT Filed: Apr. 9, 2022
(86) PCT No.: PCT/EP2022/059550

§ 371 Date: Oct. 10, 2023

(30) Foreign Application Priority Data
Apr. 10, 2021 (EP) 21167756.2

(51) Int. Cl. G01N 33/15

(52) U.S. Cl. CPC G01N 33/15; G01N 2333/91215

(57) ABSTRACT

The invention relates to a method of identifying selective covalently binding inhibitors by using a reversibly inhibiting scaffolds modified by a warhead comprising a fast-reacting Michael acceptor moiety and a linker of different length, determining kinact, and replacing the warhead of the covalently binding inhibitor with the highest kinact by a warhead comprising a moderately reacting Michael acceptor.

### CROSS-REFERENCE TO RELATED APPLICATIONS

This is the U.S. National Stage of International Patent Application No. PCT/EP2022/059550, filed Apr. 9, 2022, which claims the benefit of European Patent Application No. 21167756.2, filed Apr. 10, 2021.

### FIELD

The present invention relates to a method for identifying PI3K kinase inhibitors.

### BACKGROUND OF THE INVENTION

Phosphoisonitide 3-kinase-Akt-mammalian target of rapamycin (PI3K-Akt-mTOR) plays a central role in the control of cell growth and proliferation. Overactivation of PI3K/mTOR pathway promotes tumor cells growth and metastasis. PI3K is therefore broadly explored as therapeutic target. Many reversible pan-PI3K inhibitors investigated in clinical trials displayed a low response rate, mainly due to adverse side effects. Inhibition of PI3K using class I pan-PI3K inhibitors triggers a rapid increase in glucose and insulin blood levels.

Isoform-selective inhibition of PI3Kα might alleviate hyperglycemia and hyperinsulinemia, but the selectivity of claimed PI3Kα-specific drugs is currently limited. To date, the specific roles of the different PI3K isoforms (mainly PI3Kα and β) in insulin signaling remain controversial. A redundant physiological role of PI3Kβ in insulin action and sensitivity is under investigation. Recent studies on liver-specific p110α knockout mice showed a minor effect on hyperglycemia suggesting the rescuing role of PI3Kβ in insulin action and metabolic control when PI3Kα is inhibited.

A strategy to achieve isoform selectivity is to exploit targeted covalent inhibitors (TCIs) binding to a cysteine in or around the ATP-binding site. Most of the explored targeted cysteines are in close proximity to a reversible scaffold (e.g. for BTK and EGFR), thus a structural optimization of linker length and flexibility has not been investigated. The method of the present invention aims to optimize targeting a cysteine in or around an ATP-binding site in a protein to be inhibited.

Based on the above-mentioned state of the art, the objective of the present invention is to provide means and methods to develop optimized chemical probes that are suitable to inhibit a protein target by forming a covalent bond. This objective is attained by the subject-matter of the independent claims of the present specification, with further advantageous embodiments described in the dependent claims, examples, figures and general description of this specification.

### SUMMARY OF THE INVENTION

A first aspect of the invention relates to a method of identifying selective covalently binding inhibitors comprising the steps of
connecting a first warhead comprising a fast-reacting Michael acceptor moiety to a first scaffold that reversibly inhibits a target protein by using a first linker yielding a first covalently binding inhibitor,
connecting a second warhead comprising a fast-reacting Michael acceptor moiety to a second scaffold that reversibly inhibits said target protein by using a second linker yielding a second covalently binding inhibitor, wherein
   the first warhead and the second warhead are identical,
   the first scaffold and the second scaffold are identical,
   the first linker and the second linker differ in length,
determining K_{inact} of the first and second covalently binding inhibitor with respect to forming a covalent bond to a cysteine that is in proximity to the binding site of said first and second scaffold at said target protein,
replacing the warhead of the covalently binding inhibitor with the highest K_{inact} by a warhead comprising a moderately reacting Michael acceptor yielding a selective covalently binding inhibitor.

### TERMS AND DEFINITIONS

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

The term “enone” refers to a α,β-unsaturated carbonyl, that is a type of organic compound consisting of an alkene conjugated to a ketone. The simplest enone is methyl vinyl ketone (butenone) or CH₂═CHCOCH₃. They are electrophilic at both the carbonyl carbon as well as the B-carbon. Depending on conditions, either site is attacked by nucleophiles. Additions to the alkene are called Michael additions and are used in the present invention to covalently modify a target such as Cysteine 862 in PI3Kα.

The term “acrylamide” refers to an amide that is derived from acrylic acid and has the general chemical formula CH₂═CHC(O)NH₂. Acrylamides are used in compounds of the present invention and undergo Michael addition with Cysteine 862 in PI3Kα.

The term “PI3K” refers to phosphoinositide 3-kinase.

The term “PI3Kalpha”, “PI3Kα” or “p110a protein” relates to a subunit of PI3K that is encoded by the PI3KCA gene.

The term “irreversible” or “irreversible inhibitor” refers to an inhibitor that is able to be covalently bonded to a PI3 kinase in a substantially non- reversible manner, whereas a reversible inhibitor is able to bind to (but is generally unable to form a covalent bond with) a kinase, and therefore can be dissociated from the PI3 kinase. An irreversible inhibitor will remain substantially bound to a kinase once covalent bond formation has occurred. Methods for identifying if a compound is acting as an irreversible inhibitor are known to one of ordinary skill in the art. Such methods include, but are not limited to, enzyme kinetic analysis of the inhibition profile of the compound with the kinase, the use of mass spectrometry of the protein drug target modified in the presence of the inhibitor compound, the use of X-ray crystallography to solve the complex between the protein drug target and the inhibitor compound, discontinuous exposure, also known as “washout” experiments, as well as other methods known to one of skill in the art.

The term “warhead” or “warhead group” refers to a functional group present on a compound of the present invention wherein that functional group is capable of covalently binding to an amino acid residue (such as cysteine, lysine, histidine, or other residues capable of being covalently modified) present in the binding pocket of the target protein, thereby irreversibly inhibiting the protein. Warhead groups are essential for covalently, and irreversibly, inhibiting the protein.

The term “inhibitor” is defined as a compound that binds to and inhibits PI3 kinase with measurable affinity. In certain embodiments, the inhibitors are characterized by IC₅₀ and/or rate constant for irreversible inactivation (Kinact).

Terms such as “compound of this invention” and “compounds of the present invention” include stereoisomers, geometric isomers, tautomers, solvates, pharmaceutically acceptable salts, and solvates of the salts thereof.

The phrase “pharmaceutically acceptable salt” as used herein, refers to pharmaceutically acceptable organic or inorganic salts of a compound of the invention. If the compound of the invention is a base, the desired pharmaceutically acceptable salt may be prepared by any suitable method available in the art, for example, treatment of the free base with an inorganic acid, such as hydrochloric acid, hydrobromic acid, sulfuric acid, nitric acid, methanesulfonic acid, phosphoric acid and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

Unless defined otherwise, all technical and scientific terms used herein have the same meaning as commonly understood by one of ordinary skill in the art.

### DETAILED DESCRIPTION OF THE INVENTION

A first aspect of the invention relates to a method of identifying selective covalently binding inhibitors comprising the steps of
providing a first covalently binding inhibitor that is characterized by a first warhead comprising a fast-reacting Michael acceptor moiety, wherein the first warhead is connected via a first linker to a first scaffold that is able to reversibly inhibit a target protein by reversible binding to a binding site,
providing a second covalently binding inhibitor that is characterized by a second warhead comprising a fast-reacting Michael acceptor moiety, wherein the second warhead is connected via a second linker to a second scaffold that is able to reversibly inhibit said target protein by reversible binding to said binding site, wherein
   the first warhead and the second warhead are identical,
   the first scaffold and the second scaffold are identical,
   the first linker and the second linker differ in length,
determining K_{inact} of the first and second covalently binding inhibitor with respect to forming a covalent bond to a cysteine that is in proximity to the binding site of said first and second scaffold at said target protein,
replacing the first or second warhead of the covalently binding inhibitor with the highest K_{inact} by a third warhead comprising a moderately reacting Michael acceptor moiety yielding a selective covalently binding inhibitor.

An alternative aspect of the invention relates to a method of identifying selective covalently binding inhibitors comprising the steps of
connecting a first warhead comprising a fast-reacting Michael acceptor moiety to a first scaffold that reversibly inhibits a target protein by using a first linker yielding a first covalently binding inhibitor,
connecting a second warhead comprising a fast-reacting Michael acceptor moiety to a second scaffold that reversibly inhibits said target protein by using a second linker yielding a second covalently binding inhibitor, wherein
   the first warhead and the second warhead are identical,
   the first scaffold and the second scaffold are identical,
   the first linker and the second linker differ in length,
determining k_{inact} of the first and second covalently binding inhibitor with respect to forming a covalent bond to a cysteine that is in proximity to the binding site of said first and second scaffold at said target protein,
replacing the warhead of the covalently binding inhibitor with the highest k_{inact} by a warhead comprising a moderately reacting Michael acceptor yielding a selective covalently binding inhibitor.

In certain embodiments, the fast-reacting Michael acceptor moiety of the first or second warhead is an α,β unsaturated carbonyl, wherein
the unsaturated moiety is ethenyl or ethynyl, particularly ethenyl, and
the α position is unsubstituted, and
the β position is unsubstituted or monosubstituted.
The method according to any of the preceding claims, wherein the first or second warhead comprising a fast-reacting Michael acceptor moiety is selected from a moiety of formula 1a or 1b, particularly 1a,

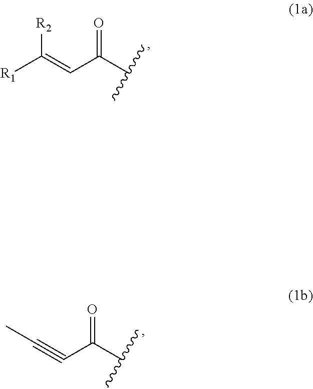

wherein
one of the moieties R¹ and R² is H and
the other moiety R² or R¹ is selected from H, CH₃, cyclopropyl, —F, —CH₂—F, —CH₂—CH₂—F, —CN, —N(CH₃)—CH₃,

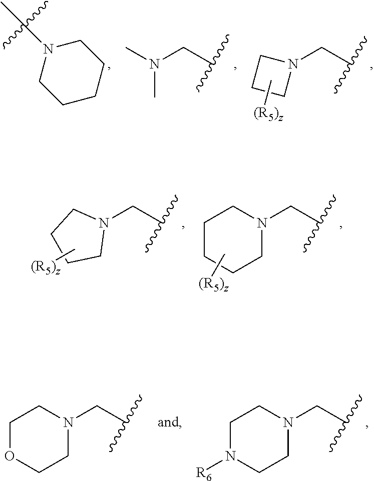

with R₅ being F or CH₃, R₆ being C₁₋₆-alkyl and z being 0, 1 or 2.

In certain embodiments, one of the moieties R¹ and R² H, and the other moiety R² or R¹ is H, CH₃,

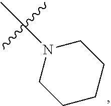 [SMILES: *N1CCCCC1]

or —N(CH₃)—CH₃, particularly H or CH₃.
The method according to any of the preceding claims, wherein the moderately reacting Michael acceptor moiety of the third warhead is an α,β unsaturated carbonyl, wherein
   the unsaturated moiety is ethenyl or ethynyl, particularly ethenyl, and
   the α position is unsubstituted, and
   the β position is fully substituted.

In certain embodiments, the third warhead comprising the moderately reacting Michael acceptor moiety is selected from a moiety of formula 2a,

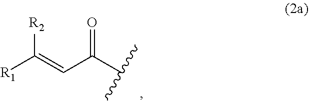

wherein
R¹ and R² are independently from each other selected from CH₃, cyclopropyl, —F, —CH₂—F, —CH₂—CH₂—F, —CN, —N(CH₃)—CH₃,

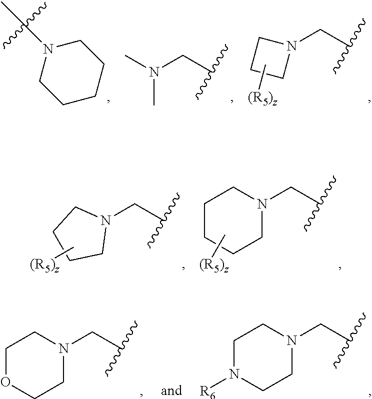

with R₅ being F or CH₃, R₆ being C₁₋₆-alkyl and z being 0, 1 or 2,
particularly R¹ and R² are independently selected from CH₃, cyclopropyl, —CN, —N(CH₃)—CH₃,

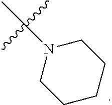 [SMILES: *N1CCCCC1]

The method according to claim 6, wherein R¹ and R² are independently selected from CH₃,

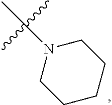 [SMILES: *N1CCCCC1]

and —N(CH₃)—CH₃, particulalry CH₃.

In certain embodiments, the first and second linker are composed of C, N, O and/or H atoms, wherein the linker has a length between 3 Å and 15 Å, particularly between 7 Å and 14 Å, more particularly between 10 Å and 13 Å, even more particularly between 10.5 Å and 12.5 Å.

In certain embodiments, the first and second linker consists of 2 to 5 moieties selected from C₁₋₆-alkyl, —CO—, —NH—, —N(CH₃)—, —O—, phenyl, and a heteroaliphatic 4-, 5- or 6-membered ring, particularly C₁₋₆-alkyl, —CO—, —NH—, —N(CH₃)—, and a heteroaliphatic 4-, 5- or 6-membered ring. In certain embodiments, the first and second linker consists of 2 to 5 moieties selected from C₁₋₆-alkyl, —CO—, —NH—, —N(CH₃)—, —O—, phenyl,

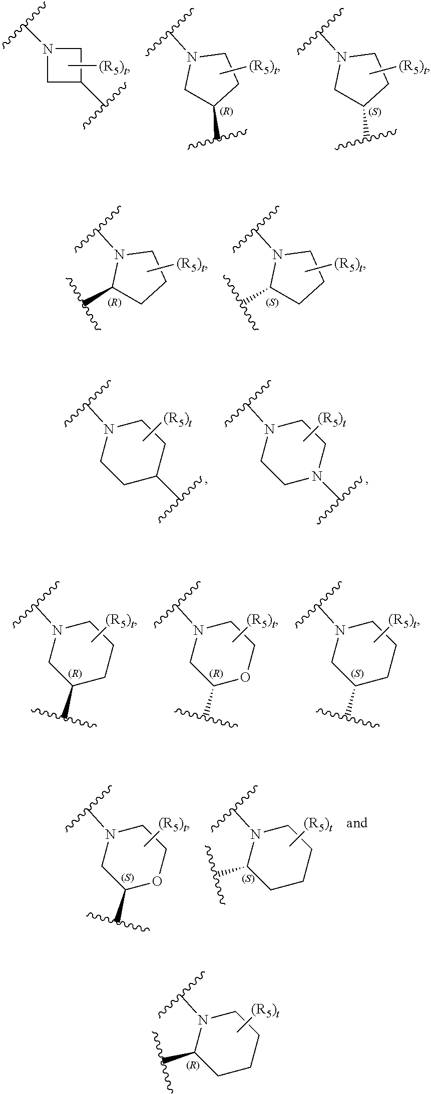

with R₅ being C₁₋₃-alkyl, F, —CH₂CN or —CN and t being 0, 1 or 2, particularly 0.

In certain embodiments, the first and second linker consists of 2 to 5 moieties selected from C₁₋₆-alkyl, —CO—, —NH—, —N(CH₃)—,

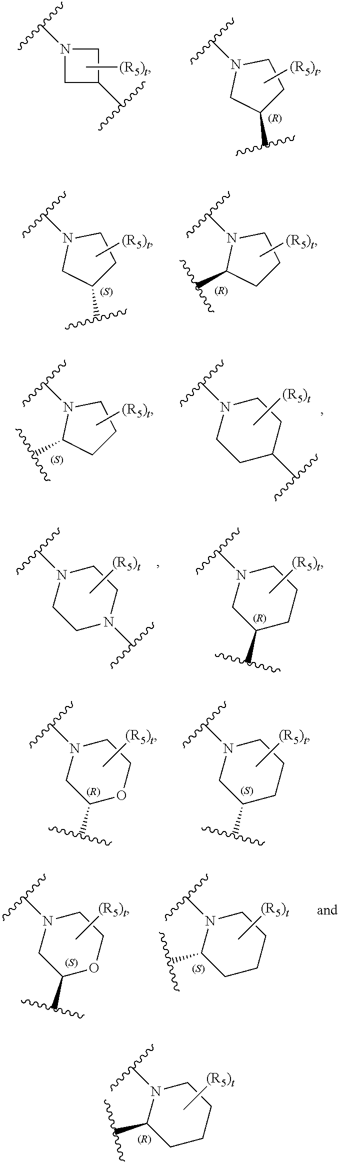

with R₅ being C₁₋₃-alkyl, F, —CH₂CN or —CN and t being 0, 1 or 2, particularly 0.

In certain embodiments, the first and second linker are selected from

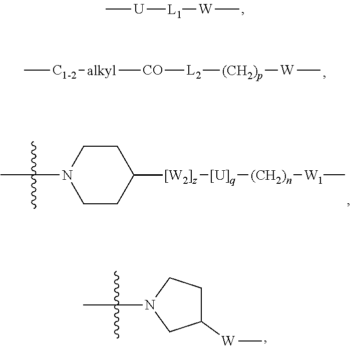

wherein
U is —CH₂— or —NH— or —N(CH₃)— or —O— or phenyl
L₁ is C₁-C₄-alkyl,
L₂ is azetidine, pyrrolidine or piperidine,
W, W₁ and W₂ are independently of each other —CO— or —CH₂—,
n is 0, 1, 2 or 3,
p, q and z are independently of each other 0 or 1.

In certain embodiments, the first and second linker are selected from L₂-W₂-U-(CH₂)ₙ-W₁-, wherein
L₂ is a moiety selected from

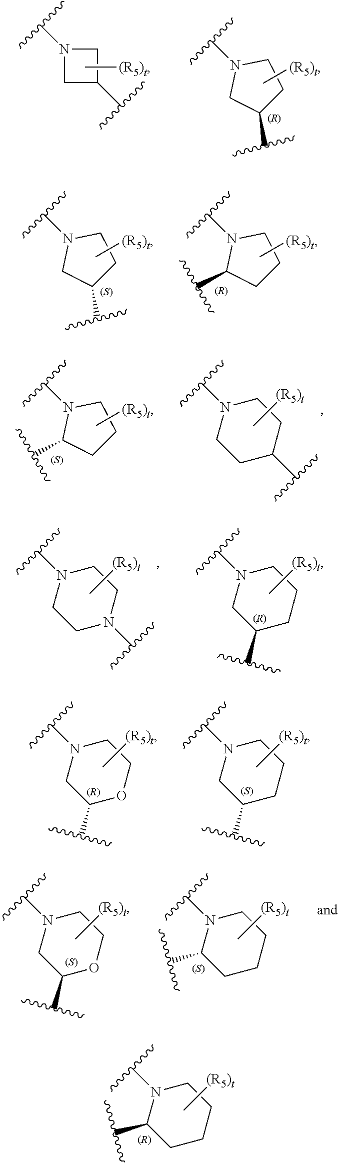

with R₅ being C₁₋₃-alkyl, F, —CH₂CN or —CN and t being 0, 1 or 2,
W₁ is CO or CH₂,
W₂ is selected from O, CH₂ and CO,
U is selected from O, CH₂, CO, NH, and N(CH₃),
n is 1 or 2.

In certain embodiments, the first and second linker are selected from
C₁₋₆-alkyl,
—CO—C₁₋₅-alkyl,
—CO—,
—C₁₋₅-alkyl-N(R)— with R being H or CH₃ or R forming a heteroaliphatic 4-, 5- or 6-membered ring,
—CO—C₁₋₄-alkyl-N(R)— with R being H or CH₃ or R forming a heteroaliphatic 4-, 5- or 6-membered ring.

In certain embodiments, the heteroaliphatic 4-, 5- or 6-membered ring is azetidine, pyrrolidine or piperidine, respectively.

In certain embodiments, the first and second scaffold are selected from a moiety of formula 3a, 3b, or 3c, particularly 3a,

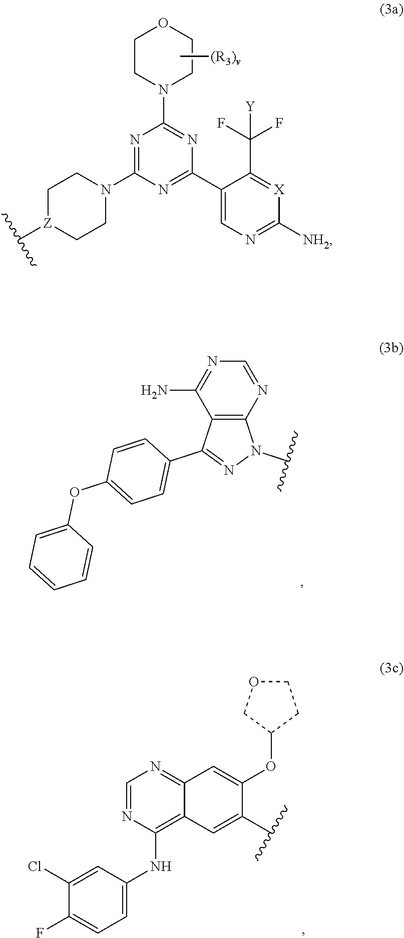

wherein
X is CH or N,
Y is Hor F,
Z is O or N, particularly N,
R₃ is C₁₋₃-alkyl or two residues R₃ form a bridge —(CH₂)ᵣ— with r being 1, 2 or 3, particularly R₃ is C₁₋₃-alkyl, more particularly CH₃,
v is 0, 1, 2, 3 or 4, particularly 0, 1 or 2, more particularly 0 or 1, even more particularly 0.

In certain embodiments, the target protein is an enzyme that comprises a cysteine in proximity to a binding site of a reversibly binding inhibitor.

In certain embodiments, the target protein is a protein kinase, particularly a tyrosine kinase.

In certain embodiments, the target protein is selected from PI3K (phosphoinositide 3-kinase), BTK (Bruton's tyrosine kinase) EGFR (epidermal growth factor receptor).

the target protein is PI3K (phosphoinositide 3-kinase), particularly PI3Kα.

The invention is further illustrated by the following examples and figures, from which further embodiments and advantages can be drawn. These examples are meant to illustrate the invention but not to limit its scope.

### DESCRIPTION OF THE FIGURES

FIG. 1 shows (a) Model of PI3Kα (gray) in complex with PQR514. (b) Superimposition of eight PI3Kα-inhibitor complexes. The position of the cysteine thiol is conserved among the different complexes (<2.6 Å distance). (c, e) Design strategy for the development of irreversible inhibitors targeting (c) BTK and (e) EGFR. The thiol group of the Cys is represented as a sphere. The distance between the reversible skeleton and the targeted cysteine is shown as dashed black lines (˜4 Å for BTK and EGFR vs ˜11 Å for PI3Kα). (d, f) Chemical structure of the reversible-inhibitor scaffolds and covalent derivatives for BTK and EGFR.

FIG. 2 shows (a) Chemical structure of a set of 9 warhead-containing compounds (1-9). (b) Chemical shift of the α-proton in 1H-NMR spectra of 3 proof-of-concept compounds. (c) Representation of LUMO and electron density surface (above) and LUMO map (below) for compound 1 using Spartan 18 Wavefunction, Inc. The LUMO map indicates where nucleophilic attack would likely occur. Colors near blue indicate high concentration of the LUMO, while colors near red indicate low concentration. (d) Chemical shift of α-proton plotted against the ELUMO values. (e) General reaction of warhead-containing compounds with -ME. (f) HPLC monitoring of loss of inhibitor (i.g. 3) and formation of the adduct with -ME (3-ME). (g) Curves displaying loss of inhibitor used to calculate k_{chem}. All values mean ±SD (n=3). (h) Intrinsic reactivity of the inhibitors (k_{chem}) plotted against the ELUMO values. (i) Model of on- and off-target covalent modification with the set of 9 compounds using KinTek.

FIG. 3 shows (a) Chemical structure of a collection of 9 compounds with pairwise matched linkers but either fast (2, 10-12) or moderate (3, 13-15) reacting Michael acceptors. (b, c, d) Comparison of (b) compounds intrinsic reactivity (k_{chem}), (c) rate constants for covalent binding to PI3Kα (k_{inact}), (d) Kᵢ. All values are shown as mean±SD (n=3). Error bars not shown when smaller than the symbols. Orange, strong electrophiles; green, weak electrophiles. The distance between the Michael acceptor (β-carbon) and Cys862 thiol was calculated using PyMOL 2.3.5 Schrödinger, Inc. (e) Time-dependent TR-FRET experiments for compounds with a strong electrophile.

FIG. 4 shows (a) Library of acrylamide-containing compounds (16-24). (b) Intrinsic reactivity (k_{chem}) of compounds 16-24, ibrutinib and CNX-1351. Experiments performed with HPLC (1 mM inhibitor and 600 mM -ME, n=3). All values are reported as mean±SD. (c) Effciency in covalent bond formation (k_{inact}/Kᵢ) plotted against distance from Cys862. Zero (x-axis) correspond to Cys862 positioning; left to zero: shorter linkers; right to zero: longer linkers. (d) The electron density clearly shows that the acrylamide of 19 forms a covalent bond with the thiol group of Cys862.

FIG. 5 shows (a, b, c, d) BRET experiments using HEK293 cells expressing NanoLuc fused (a) PI3K, (b) PI3K C862S, (c) PI3K- and (d) PI3Kα. Cells were incubated with inhibitors (3 μM) for 2 hours, washed out and treated with the tracer. Displacement of the inhibitors was monitored by BRET. Data shown are means±SD (n=3).

FIG. 6 shows (a) Cellular washout studies in SKOV3 cells. The experiments were performed in triplicate; CNX-1351 was used as positive control (irreversible inhibitor) and PQR514 as negative control (reversible inhibitor). (b) Metabolic stability of 19, 22 and CNX-1351 using rat liver microsomes. The time-dependent loss of test items (1 μM) with liver microsomes (0.5 mg microsomal protein/mL) and the percentages of test item remaining after 60 minutes of incubation were measured, (n=2).

FIG. 7 shows the chemical structure of PI3Ks reversible inhibitors PQR309 and PQR514. General structure of β,β-dimethyl enone warhead covalent inhibitors (general structure of selected linkers are shown in blue and CNX-1351 warhead in red).

FIG. 8 Crystal structure of compound 7b in PI3Kα(green). Major interactive residues are shown.

FIG. 9 shows cellular washout studies in SKOV3 cells. The experiments were performed in triplicate; CNX-1351 was used as positive control (irreversible inhibitor) and PQR514 as negative control (reversible inhibitor).

FIG. 10 Structure of PI3Kα-selective covalent inhibitor 1 and its direct reversible analog r-1 lacking a Michael acceptor on the warhead. (b) X-ray crystallographic structure of PI3Kαin complex with one of our covalent inhibitor. (c-d) TR-FRET experiments of (c) a proof-of-concept covalent binder (1) and (d) its non-covalent derivative (r-1).

FIG. 11 shows Characterization of binding mode in HEK293 cells using NanoBRET platform. (a) A schematic representation of NanoBRET drug displacement assay platform. There is a BRET event occurs between NanoLuc (donor) and the tracer (acceptor) when the drug is displaced from the binding pocket by the tracer. After drug washout, prolonged residence time of inhibitor on the target indicates irreversible binding on the target. (b) Class I PI3K isoforms alignment indicating non-conserved C862 in PI3Kα can be targeted selectively. (c) Prolonged residence time of 1 compared to its reversible analog r-1 indicates irreversible binding of 1 on the PI3Kα isoform. (c) Introduction of a genetic point mutation of PI3Kα C862S diminishes prolonged residence time of 1 and validates C862 involvement in covalent modification. (e, f) 1 and r-1 do not show prolonged residence time on PI3Kβ and PI3Kδ isoforms after washout; no off-target covalent modification is detected. Data shown are means±SEM (n=3).

FIG. 12 shows Intracellular drug-target engagement kinetic studies revealed gain in potency due to covalent targeting and optimized cellular permeability in HEK293 cells. (a) A schematic representation of NanoBRET tracer displacement assay platform. Drug binding to the target displaces the tracer from the ATP binding pocket and diminishes the BRET event between NanoLuc (donor) and the tracer (acceptor). Drug-target engagement kinetics is measured in HEK293 cells for compounds (b) 1 and (c) CNX1351 (d) Multidimensional characterization of intracellular drug binding kinetics based on correlating diffusion coefficient of the compounds with their covalent bond formation efficiencies inside intact HEK293 cells. Data shown are means±SEM (n=3). Error bars not shown when smaller than the symbols.

FIG. 13 shows (a, b, c, d) Deconvolution of class IA isoform action using isoform selective reversible PI3K inhibitors including BYL719 (PI3Kα), TGX221 (PI3Kβ), CAL101 (PI3Kδ), and PI3Kα-selective covalent inhibitor, 1 in PI3Kα inhibition sensitive (a) T47D (PIK3CA H1047R) and (b) MCF7 (PIK3CA E545K) and resistant (c) PC3 (PTEN-deficient) and (d) A2058 (PTEN-deficient/BRAF V600E) cell lines. Transient pan-PI3K/covalent PI3Kα targeting of 1 introduces gain in potency over the isoform selective reversible inhibitors in continuous drug treatment. (e, f, g, h) Cellular washout studies in PI3Kα inhibition sensitive (e) T47D and (f) MCF7 and resistant (g) PC3 and (h) A2058 cell lines. (e, f) 1 introduced prolonged inhibition of Akt (S473) phosphorylation in the PI3Kα inhibition-sensitive cell lines; however, (g, h) contribution of other class IA isoforms partially reactivate Akt (S473) phosphorylation in the resistant cell lines after drug washout. All the reversible inhibitors including r-1 lose their inhibitory activity after washout. Akt (S473) phosphorylation level below the dashed line (red) indicates contribution of other class I PI3K isoforms while PI3Kα isoform is covalently inhibited by 1. Data shown are means±SEM (n=4). Error bars not shown when smaller than the symbols.

FIG. 14 shows Dynamic probing of PI3Kα-selective prolonged inhibition in cancer cells. FOXO1 cytosolic/nuclear translocation was used as a real time readout for PI3K/Akt activation. Long-lasting efficacy of compound 1 in PI3Kα-driven cancer cells and dynamic probing of PI3K signaling using FOXO1 cytosolic/nuclear translocation sensors as a real time readout for PI3K/Akt activation. (a, b) Time course of cytosol:nuclear ratio of FOXO1-Clover in (c) T47D and (d) A2058 cells treated with 3 μM 1 and 3 μM BYL719, or DMSO for 1 hour following drug addition and (c, d) 10 hours following drug washout. Representative images of (e) T47D and (f) A2058 from time course experiments.

FIG. 15 shows Prolonged anti-proliferative activity of intermittent dosing in cancer cells. (a) Dose-response correlation with varying concentration of compounds, 1, r-1, and BYL719 for growth inhibition in T47D, MCF7, and A2058 cells after 72 h continuous drug treatment. (b) Effect of intermittent drug treatment to growth proliferation. 5 μM compound is treated for 4 h a day repeated for 4 days in T47D, MCF7, and A2058. 1 showed prolonged anti-proliferative activity compared to reversible inhibitors r-1 and BYL719. Data shown are means±SEM (n≥3).

### EXAMPLES

### Example 1: Volume Scanning, a Rational Approach to Covalent PI3Kα Inhibitors

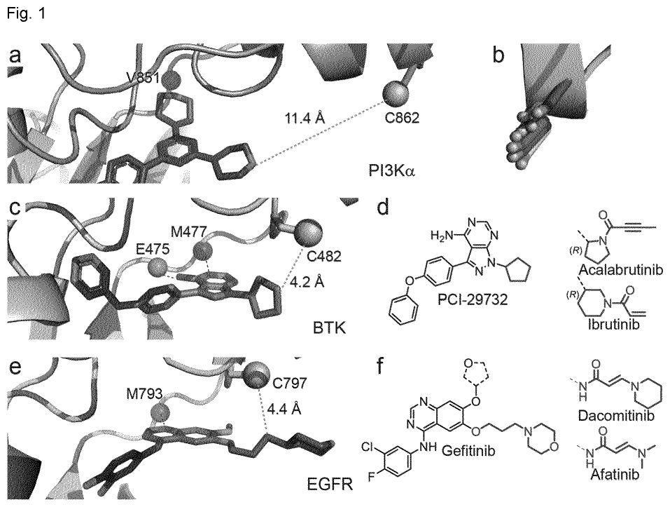

A strategy to achieve isoform selectivity is to exploit targeted covalent inhibitors (TCIs) binding to a cysteine in or around the ATP-binding site. Most of the explored targeted cysteines are in close proximity to the reversible scaffold (e.g. for BTK and EGFR, FIG. 1D and 1F), thus a structural optimization of linker length and flexibility has not been investigated. The present invention aims to target the non-conserved Cys862 in PI3Kα, which is located at >10 Å from the core reversible compound (PQR309[1] and PQR514[4]).

### Fine-Tuning Warhead Reactivity

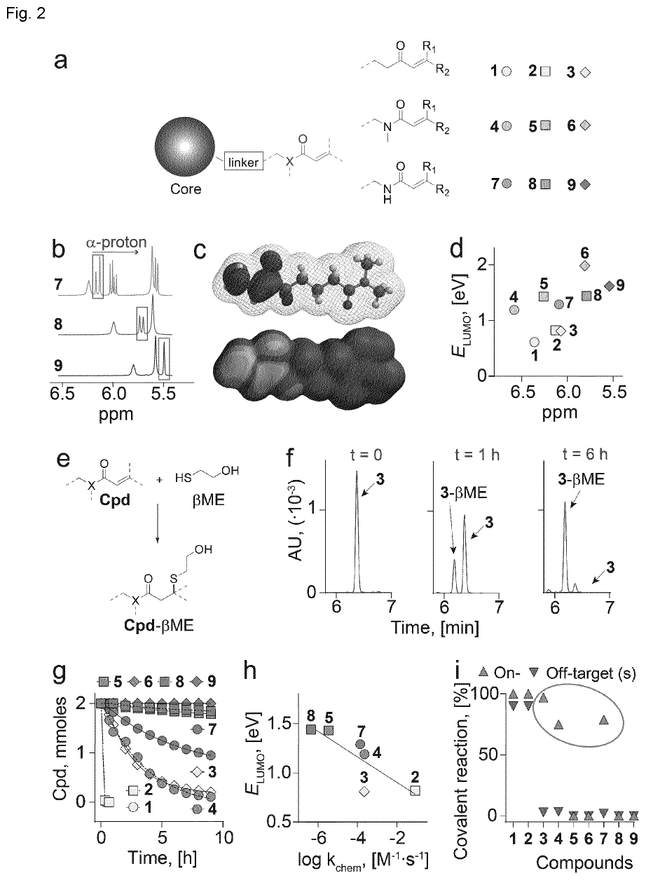

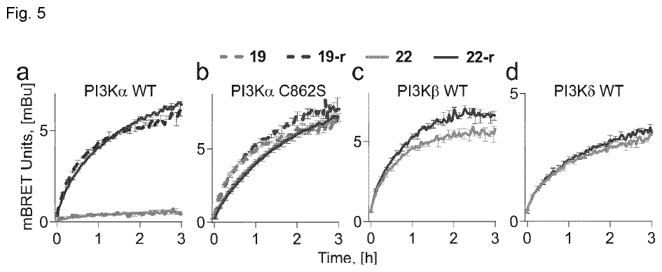

The understanding of warhead reactivity is a pivotal parameter in the development of covalent compounds as drugs candidates and chemical probes. Herein, we designed and synthesized a set of compounds bearing nine different warheads, including enones and acrylamides (1-9, FIG. 2A). The lowest-unoccupied molecular orbitals (LUMO) value was calculated for each compound (FIG. 2C). The chemical shifts at the α position in 1H NMR is not a reliable predictor of reactivity when comparing structurally different chemical entities. Therefore, we set up a method to measure the reaction rate constant between the diverse set of Michael acceptors and β-mercaptoethanol (βME, FIG. 2E). The progress of the reaction was monitored by HPLC-MS (FIG. 5F). The curves displaying the loss of inhibitors (FIG. 2G) were used to calculate the reaction rate constants (k_{chem}). The experimental measurement of k_{chem} is essential for a systematic classification of warheads reactivity. Considering that the concentration of intracellular glutathione (GSH) is approx. 7 mM, KinTek Global Kinetic Explorer was used to model the on-target (PI3Kα) and off-target (GSH and other cellular thiols) reactions. This model allowed to identify (i) highly reactive electrophiles (1, 2) which react with the target as well as with off-targets; and (ii) moderate electrophilic moieties (3, 4, 7) that selectively bind the selected target (FIG. 21). Moderately electrophilic groups should be exploited in a lead optimization project to minimize side reactions with off-target cysteines and GSH, and to increase the possibilities of success of covalent drugs. On the contrary, high electrophilicity can be exploited in the first stages of hit identification to rapidly convert a reversible drug into a covalent inhibitor and to investigate the druggability of the targeted cysteine.

### Scan of Protein Space

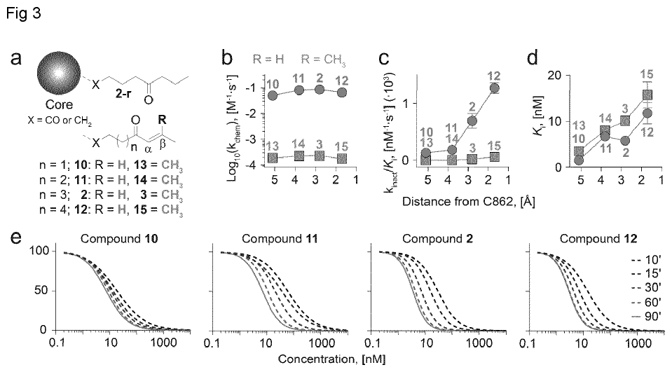

The B-methyl substituted enone (2) and its corresponding moderately electrophilic derivative, β,β-dimethyl enone (3) were selected to explore the target space. A collection of compounds with pairwise matched linkers but either fast (2, 10-12) or moderate (3, 13-15) reacting Michael acceptors were synthesized (FIG. 3A). A time-dependent inhibition (IC₅₀ shift) assay with five pre-incubation times was designed to identify covalent, time-dependent inhibitors. For compounds bearing a fast-reacting warhead (2, 10-12) a significant IC₅₀ shift was observed also for compounds having their Michael acceptor at >4 Å from the cysteine thiol (FIG. 3E). Compound 12 showed the highest maximum potential rate of inactivation (k_{inact}/Kᵢ=1.27×10⁻² nM⁻¹·s⁻¹). While the increase in linker length did not influence the intrinsic reactivity of the inhibitors for both fast and moderate reacting Michael acceptors (FIG. 3B), it significantly affected the kinact for strong electrophiles (FIG. 3C). The increased efficiency in covalent bond formation for 12 is due to the irreversible binding rather than an improvement in reversible affinity (see Kᵢ values in FIG. 3D). Thus, a fast-reactive warhead is the best option to rapidly explore the target space and to access the feasibility to target a specific distal cysteine.

### Drug-Like Warheads—Importance of Positioning

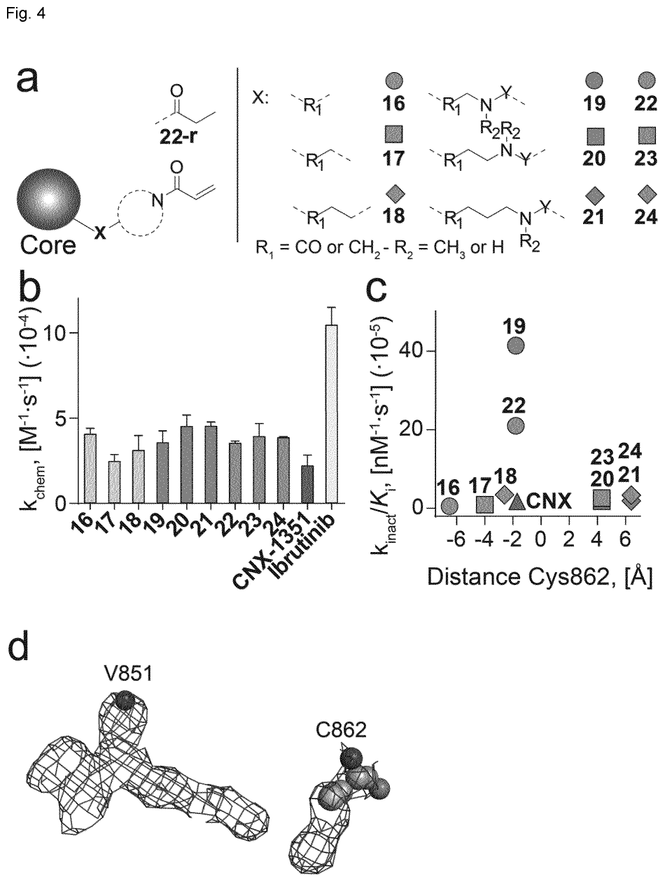

Unsubstituted acrylamides could be exploited in TCIs with the advantage of avoiding off-target reactions with other nucleophiles into the cells. Therefore, nine molecules bearing an acrylamide group and different linkers (FIG. 4A) were synthesized aiming to investigate the spatial trajectory for Cys862 targeting. The data proved that the excellent efficiency of compounds 19 and 22 in covalently modifying PI3Kα is related to the optimal positioning of the warhead driven by the linker length rather than to an increase in intrinsic reactivity (FIG. 4B and 4C). Compounds 19 and 22 displayed k_{inact}/Kᵢ values one order of magnitude higher than that of CNX-1351 [5], being 24- and 12-fold more efficient.

### NanoBRET Assay—Drug-Target Engagement in Living Cells

The inventors have examined the irreversible covalent behavior of 19 and 22 in HEK293 cells expressing NanoLuc fused PI3Ks (PI3Kα, PI3Kα C862S, PI3Kβ and PI3Kδ). The reversible analogs of 19 and 22 (19-r and 22-r) were included as negative controls. In washout experiments with wild type PI3Kα, no recovery of the BRET signal was observed for both compounds 19 and 22 (FIG. 5A). On the contrary, washout experiments using PI3Kα mutant C862S displayed comparable dissociation rates for 19, 22, and their corresponding reversible analogs 19-r and 22-r (FIG. 5B). Recovery of BRET signal was achieved also after drug washout in PI3Kβ and PI3Kδ (FIG. 5C and 5D). Therefore, the NanoBRET assays demonstrated that compounds 19 and 22 selectively and irreversibly modify PI3Kα in cells, by targeting Cys862.

### Cellular Washout Experiments and Metabolic Stability

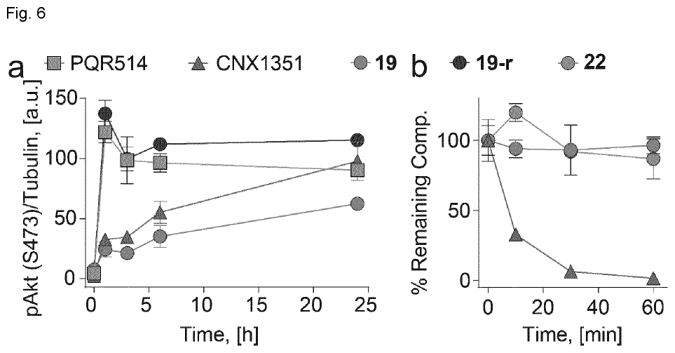

The library of acrylamide-containing compounds was evaluated for PI3K signaling in SKOV3 ovarian cancer cells. Compounds 19 and 22 displayed a good cellular activity, being twice more potent in cells than CNX-1351. Inhibition of AktSer473 phosphorylation measured by immunoblot analysis was used as an indicator of inhibition of PI3K signaling. Cells were incubated with the drugs (2.5 μM each) for 2 hours. After drug washout, the signaling was immediately recovered for PQR514 and 19-r. On the contrary, a prolonged inhibition of P-AktSer473 was observed for 19, as well as for CNX-1351 (FIG. 6A).

The metabolic stability of 19, 22 and CNX-1351 was evaluated in vitro in rat liver microsomes. Compounds 19 and 22, bearing a moderate-reactive, drug-like warhead, as well as an amide-containing linker, outperformed the rapidly metabolized CNX-1351 (FIG. 6B).

### Example 2: Development of Optimized Chemical Probes Targeting PI3Ka to Deconvolute the Role of Class I PI3Ks Isoforms in Insulin Signaling

Phosphoisonitide 3-kinase-Akt-mammalian target of rapamycin (PI3K-Akt-mTOR) plays a central role in the control of cell growth and proliferation. Overactivation of PI3K/mTOR pathway promotes tumor cells growth and metastasis. PI3K is therefore broadly explored as therapeutic target. Many reversible pan-PI3K inhibitors investigated in clinical trials displayed a low response rate, mainly due to adverse side effects. Inhibition of PI3K using class I pan-PI3K inhibitors triggers a rapid increase in glucose and insulin blood levels.

Isoform-selective inhibition of PI3Kα might alleviate hyperglycemia and hyperinsulinemia, but the selectivity of claimed PI3K α-specific drugs is currently limited. CNX-1351 is the only known PI3Kα inhibitor, having a limited efficiency in covalent bond form, poor in vitro and cellular potency, and chemical features not suitable for a lead optimization process. Recent studies on liver-specific p110α knockout mice showed a minor effect on hyperglycemia suggesting the rescuing role of PI3Kβ in insulin action and metabolic control when PI3Kα is inhibited.

### A Covalent Strategy to Improve Isoform Selectivity

Poor-quality and insufficiently selective molecules currently lead to misleading results, and impair reproducibility and robustness of scientific findings. The lack of selectivity of chemical probes for biological studies also applies to many marketed drugs that cannot be exploited for a detailed mechanism-based biological investigation.[3] Generation of high-quality PI3Kα chemical probes would dissect the role of PI3Ka in cancer and metabolism. Covalent inhibitors, permanently blocking target functions, have emerged as a promising strategy to enhance the ligand binding selectivity for proteins in the same family, representing ideal tools to investigate the specific roles of PI3Ka isoform. A strategy to achieve isoform selectivity is to exploit targeted covalent inhibitors (TCIs) binding to a cysteine in or around the ATP-binding site. Most of the explored targeted cysteines are in close proximity to the reversible scaffold. The inventors aim to target the non-conserved Cys862 in PI3Kα, which is located at >10 Å from the core reversible compound (FIGS. 1a and 1b, PQR309 [4] and PQR514 [5]).

### Compound Design and Library Extension

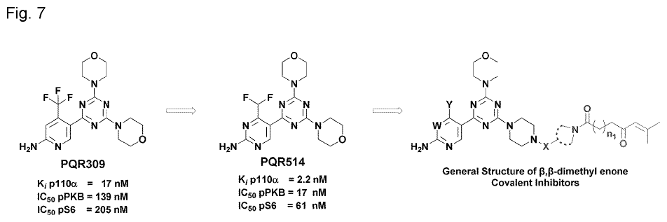

The reversible scaffold of PQR309[4] and PQR514[5] was converted into irreversible compounds. An extensive Structure Activity Relationship (SAR) study was performed using CNX-1351[1] reacting group (warhead). A collection of 12 compounds has been prepared introducing different linkers, bearing heteroaliphatic 4-,5-, and 6-member rings (FIG. 7). A time-dependent inhibition (IC50 shift) assay with five pre-incubation times was designed to identify covalent, time-dependent inhibitors.

The library was tested for cellular potency on inhibition of PKB phorphorilation, considered as an indicator of inhibition of PI3K signaling. Time-resolved fluorescence resonance (TR-FRET) tracer displacement assay was used to determine the rate constant for irreversible inactivation (k_{inact}) and the second order rate constant typically used to characterize the covalent binding of irreversible inhibitors to the target protein (k_{inact}/Kᵢ). The pilot chemical probes exceeded in vitro and cellular potency over CNX-1351. Compound 10 showed the best cellular activity, >40-fold compared to CNX-1351 (IC₅₀ for pPKB=6.69 nM vs. 290 nM). The highest maximum potential rate of inactivation (k_{inact}/K_{I}=2.60×10⁻³ nM⁻¹·s⁻¹) was also observed for compound 10, resulting in the best irreversible inhibitor of our collection, with a clogP of 2.46 compared to CNX-1351 clogP of 4.40, providing better solubility.

TABLE 1
PKB phosphorylation on Ser473 was analyzed in SKOV3 cells exposed to
the indicated inhibitors and subsequent detection of phosphoproteins
in an in-cell Western assay. Each experiment performed n = 3.
TR-FRET rate constant for irreversible inactivation (k_{inact})
and efficiency of covalent bond formation (k_{inact}/Kᵢ).

| | | | | pPKB | | |
|---|---|---|---|---|---|---|
| | | | | S473 | k_{inact}, | k_{inact}/Kᵢ, |
| Comp. | clogP | n₁ | X | IC₅₀ [nM] | [s⁻¹] | [nM⁻¹s⁻¹] |
| CNX-1351 | 4.40 | — | — | 290 | 6.57 × 10⁻⁴ | 1.73 × 10⁻⁵ |
| 1 | 2.32 | 1 | CO | 65.21 | 6.75 × 10⁻⁴ | 7.01 × 10⁻⁵ |
| 2 | 3.04 | 1 | CH₂ | 46.43 | 6.96 × 10⁻⁴ | 5.02 × 10⁻⁵ |
| 3 | 2.04 | 1 | CO | 23.67 | 6.12 × 10⁻⁴ | 5.89 × 10⁻⁵ |
| 4 | 2.04 | 1 | CO | 16.78 | 9.19 × 10⁻⁴ | 1.60 × 10⁻⁵ |
| 5 | 1.77 | 1 | CO | 33.61 | 9.12 × 10⁻⁴ | 1.29 × 10⁻⁵ |
| 6 | 2.19 | 1 | CH₂ | 33.36 | 7.32 × 10⁻⁴ | 5.16 × 10⁻⁵ |
| 7 | 2.74 | 2 | CO | 30.89 | 8.01 × 10⁻⁴ | 8.21 × 10⁻⁵ |
| 8 | 3.46 | 2 | CH₂ | 43.16 | 1.27 × 10⁻⁴ | 4.21 × 10⁻⁴ |
| 9 | 2.46 | 2 | CO | 22.69 | 2.54 × 10⁻³ | 1.71 × 10⁻⁴ |
| 10 | 2.46 | 2 | CO | 6.69 | 2.34 × 10⁻³ | 2.60 × 10⁻⁴ |
| 11 | 2.19 | 2 | CO | 11.62 | 2.15 × 10⁻³ | 1.93 × 10⁻⁴ |
| 12 | 2.85 | 2 | CH₂ | 17.31 | 1.46 × 10⁻³ | 8.69 × 10⁻⁵ |

### C862 as the Targeted Nucleophilic Residue

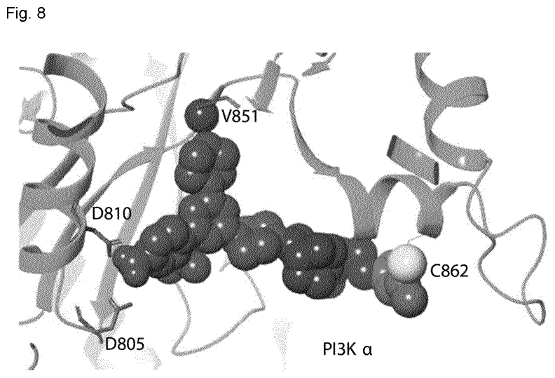

The covalent bond adducts between selected compounds and the targeted cysteine residue was confirmed by X-ray crystallography (FIG. 8). The β,β-dimethyl is clearly seen in the electron density to form a covalent bond with Cys862. The core molecule maintained the major interactions observed for the PI3Kγ-PQR309 complex[4]. The morpholine oxygen atom establishes a H-bond with the backbone amide of Val851. The NH2-group of the aminopyrimidine forms H-bond interactions with Asp805/810. In addition, bottom-up LC-MS/MS based proteomics further proved the covalent modification of Cys862 in PI3Kα.

### Cellular Washout Experiments

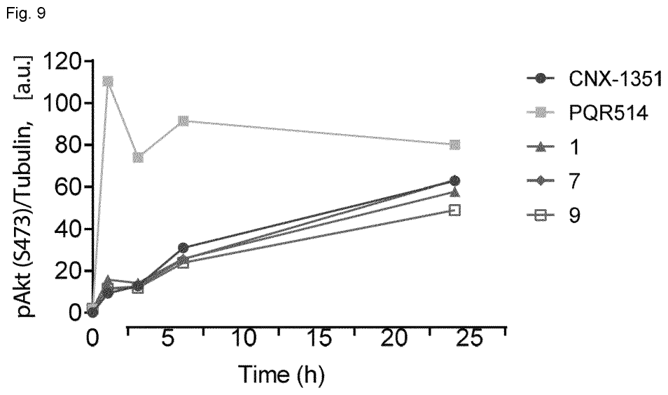

The library of enone-containing compounds was evaluated for PI3K signaling in SKOV3 ovarian cancer cells. Compounds 1, 7 and 9 displayed a good cellular activity, being 4-, 9- and 10-fold more potent in cells than CNX-1351 (Table 1), respectively. Inhibition of AktSer473 phosphorylation measured by immunoblot analysis was used as an indicator of inhibition of PI3K signaling. Cells were incubated with the drugs (2.5 μM each) for 2 hours. After drug washout, the signaling was immediately recovered for PQR514. On the contrary, a prolonged inhibition of P-AktSer473 was observed for compounds 1, 7 and 9, as well as for CNX-1351 (FIG. 9).

### Example 3: A Novel, Highly Potent PI3Kα Covalent Inhibitor Deconvolutes Class I PI3K Isoforms in Cancer Cells

Phosphoinositide-3-kinase (PI3K) signaling is a key regulator of cellular processes such as cell growth, proliferation and metabolism. Constitutively activated PI3K is frequent in tumors and drives cancer progression. PI3K is therefore broadly explored as therapeutic target, but many pan-PI3K inhibitors displayed a low response rate in clinical trials. Inhibition of PI3K using class I pan-PI3K inhibitors triggers a rapid increase in blood glucose and insulin. Recent studies on liver-specific p110α knockout mice showed a minor effect on hyperglycemia suggesting the rescuing role of PI3Kβ in insulin action and metabolic control when PI3Kα is inhibited. The inventors developed a rational approach to increase target selectivity by exploiting a covalent binding of inhibitors targeting non-conserved Cys862 residue on PI3Kα.

### PI3Kα-Selective Covalent Inhibition Strategy

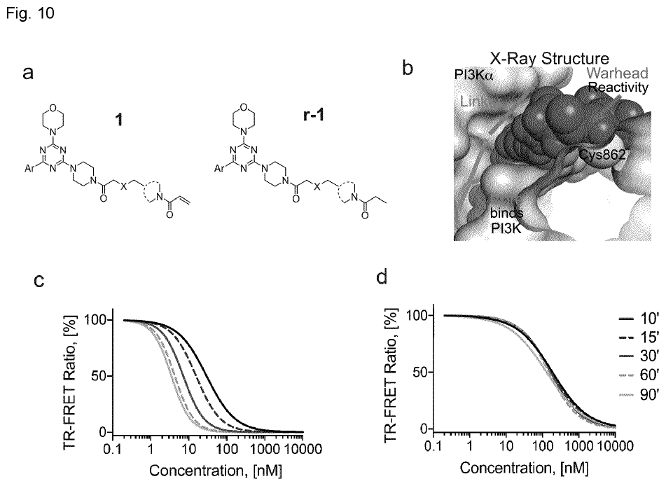

Non-conserved C862 residue in PI3Kα is targeted to irreversibly inhibit PI3Kα activity by exploiting covalent inhibition strategy. The X-ray crystallographic structure of PI3Kαin complex with one of our covalent inhibitor was solved, proving the covalent bond between the thiol group of Cys862 and the warhead. Moreover, to confirm the covalent nature of the PI3Kα inhibitors, the inventors have synthesized the corresponding reversible analogs and analyzed irreversible/reversible analogs using a wide range of biochemical and cellular assay platforms. TR-FRET assay was designed to measure drug-target engagement kinetics on the recombinant protein. Only the irreversible inhibitor presented an IC₅₀-shift but not its reversible analog, which is a typical feature of TCIs (FIG. 10).

### Multidimensional Approach to Characterize Covalent Inhibitors

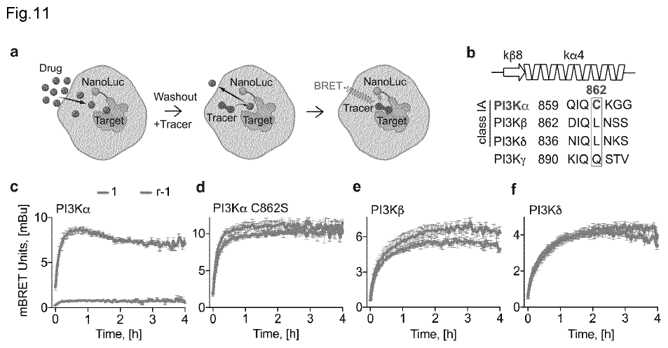

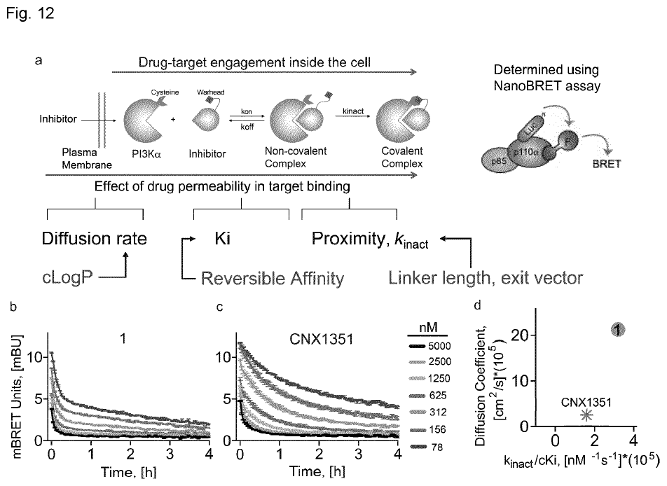

Establishment of a NanoBRET cellular assay for quantification of drug-target engagement kinetics inside live cells introduces a new platform to get physiologically relevant drug-target engagement profile. NanoBRET assay platform exploiting the PI3Kα C862S mutation confirmed the covalent modification of Cys862. In addition, it has been shown that only the targeted PI3Kα is covalently modified by the compound 1 among class IA PI3K isoforms (FIG. 11). Moreover, intracellular drug-target engagement kinetic studies revealed superior cellular permeability and drug-target engagement profiles of our lead covalent inhibitor 1 compared to CNX1351 (FIG. 12).

### Prolonged Inhibition in PI3Kα-Driven Cancer Cells

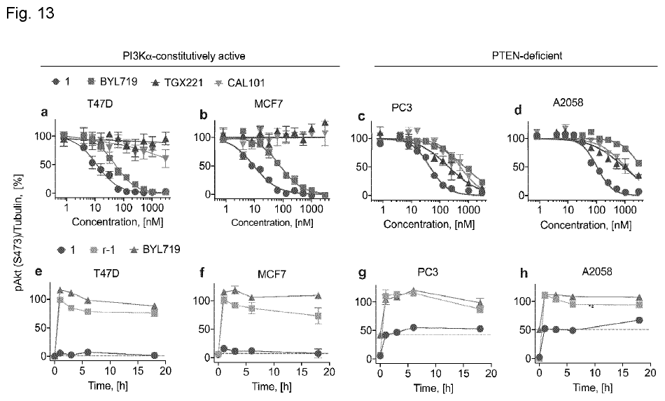

Class IA PI3K isoform signaling activities are deconvoluted in terms of phosphorylation of its downstream effector, Akt (S473) using isoform selective reversible PI3K inhibitors. Accordingly, drug washout studies using 1 indicated novel targeting strategies by promoting prolonged PI3Kα inhibition in cancer cells. Irreversible elimination of PI3Kα activity was efficacious to downregulate PI3K aberrant signaling in PI3Kα-driven cancer cells. In the PTEN-deficient cancers, residual Akt phosphorylation after drug washout indicated redundant roles of class IA PI3K isoforms. (FIG. 13)

### Dynamic Probing of PI3Kα-Selective Prolonged Inhibition

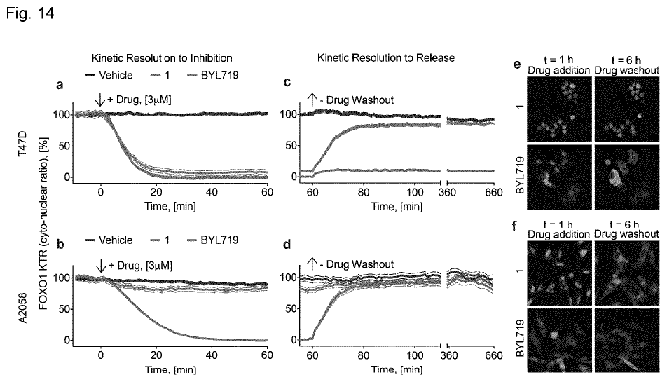

In order to study dynamics of PI3K/Akt downstream activity at the single cell level, stable cell lines expressing a fluorescent reporter fused-Forkhead box protein O1 (FOXO1) has been incorporated, which translocates from the nucleus to the cytoplasm in response to Akt stimulation. Dynamic translocation probes based on the FOXO1 transcription factor enabled measurement of signaling downstream of Akt and revealed on/off dynamics of the PI3K/Akt signaling axis. (FIG. 14)

### Prolonged Anti-Proliferative Activity

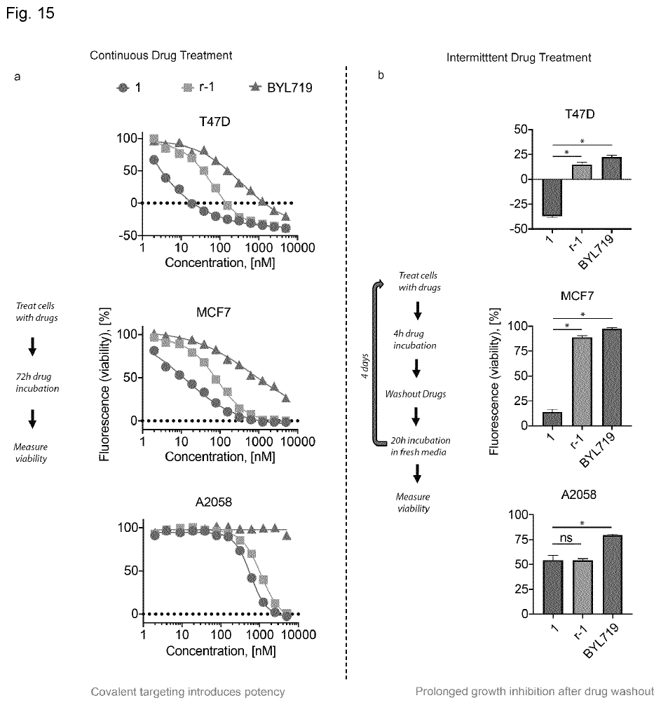

PI3Kα-selective covalent inhibition strategy introduced gain in potency and prolonged inhibitory activity in terms of growth inhibition of PI3Kα-constitutively active luminal breast cancer cell lines. In PTEN deficient melanoma cell line (A2058), pan-PI3K/PI3Kα-selective covalent inhibition strategy using 1 showed superior inhibitory activity compared to PI3Kα-selective reversible inhibition using BYL719. (FIG. 15)

1. A method of identifying selective covalently binding inhibitors comprising the steps of
providing a first covalently binding inhibitor that is characterized by a first warhead comprising a fast-reacting Michael acceptor moiety, wherein the first warhead is connected via a first linker to a first scaffold that is able to reversibly inhibit a target protein by reversible binding to a binding site,
providing a second covalently binding inhibitor that is characterized by a second warhead comprising a fast-reacting Michael acceptor moiety, wherein the second warhead is connected via a second linker to a second scaffold that is able to reversibly inhibit said target protein by reversible binding to said binding site, wherein
the first warhead and the second warhead are identical,
the first scaffold and the second scaffold are identical,
the first linker and the second linker differ in length,
determining k_{inact} of the first and second covalently binding inhibitor with respect to forming a covalent bond to a cysteine that is in proximity to the binding site of said first and second scaffold at said target protein,
replacing the first or second warhead of the covalently binding inhibitor with the highest k_{inact} by a third warhead comprising a moderately reacting Michael acceptor moiety yielding a selective covalently binding inhibitor.

2. The method of claim 1, wherein the fast-reacting Michael acceptor moiety of the first or second warhead is an α,β unsaturated carbonyl, wherein
the unsaturated moiety is ethenyl or ethynyl, particularly ethenyl, and
the α position is unsubstituted, and
the β position is unsubstituted or monosubstituted.

3. The method according to claim 1, wherein the first or second warhead comprising a fast-reacting Michael acceptor moiety is selected from a moiety of formula 1a or 1b, particularly 1a, 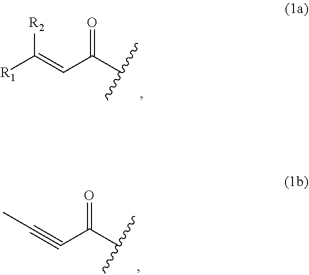
wherein
one of the moieties R¹ and R² is H and
the other moiety R² or R¹ is selected from H, CH₃, cyclopropyl, —F, —CH₂—F, —CH₂—CH₂—F, —CN, —N(CH₃)—CH₃, 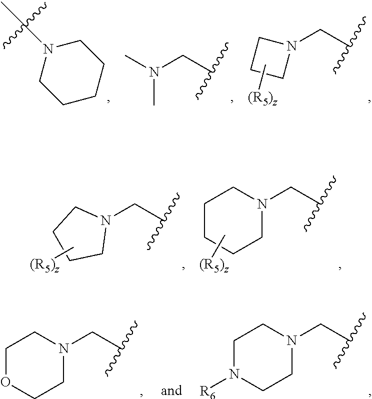
with R₅ being F or CH₃, R₆ being C₁₋₆-alkyl and z being 0, 1 or 2.

4. The method according to claim 3, wherein one of the moieties R¹ and R² H, and the other moiety R² or R¹ is H, CH₃, 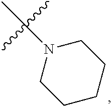 [SMILES: *N1CCCCC1]
or —N(CH₃)—CH₃, particularly H or CH₃.

5. The method according to claim 1, wherein the moderately reacting Michael acceptor moiety of the third warhead is an α,β unsaturated carbonyl, wherein
the unsaturated moiety is ethenyl or ethynyl, particularly ethenyl, and
the α position is unsubstituted, and
the β position is fully substituted.

6. The method according to claim 1, wherein the third warhead comprising the moderately reacting Michael acceptor moiety is selected from a moiety of formula 2a, 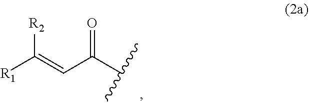
wherein
R¹ and R² are independently from each other selected from CH₃, cyclopropyl, —F, —CH₂—F, —CH₂—CH₂—F, —CN, —N(CH₃)—CH₃, 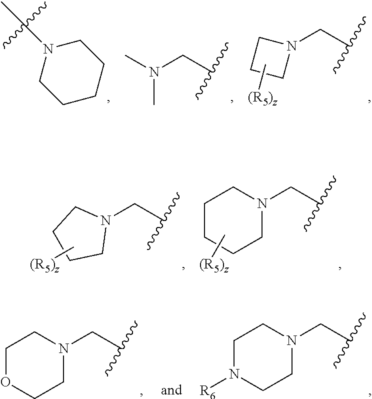
with R₅ being For CH₃, R₆ being C₁₋₆-alkyl and z being 0, 1 or 2,
particularly R¹ and R² are independently selected from CH₃, cyclopropyl, —CN, —N(CH₃)—CH₃,

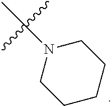 [SMILES: *N1CCCCC1]

7. The method according to claim 6, wherein R¹ and R² are independently selected from CH₃, 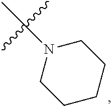 [SMILES: *N1CCCCC1]
and —N(CH₃)—CH₃, particulalry CH₃.

8. The method according to claim 1, wherein the first and second linker are composed of C, N, O and/or H atoms, wherein the linker has a length between 3 Å and 15 Å, particularly between 7 Å and 14 Å, more particularly between 10 Å and 13 Å, even more particularly between 10.5 Å and 12.5 Å.

9. The method according to claim 1, wherein the first and second linker consists of 2 to 5 moieties selected from C₁₋₆-alkyl, —CO—, —NH—, —N(CH₃)—, —O—, phenyl, and a heteroaliphatic 4-, 5- or 6-membered ring, particularly C₁₋₅-alkyl, —CO—, —NH—, —N(CH₃)—, and a heteroaliphatic 4-, 5- or 6-membered ring.

10. The method according to claim 1, wherein the first and second linker consists of 2 to 5 moieties selected from C₁₋₆-alkyl, —CO—, —NH—, —N(CH₃)—, —O—, phenyl, 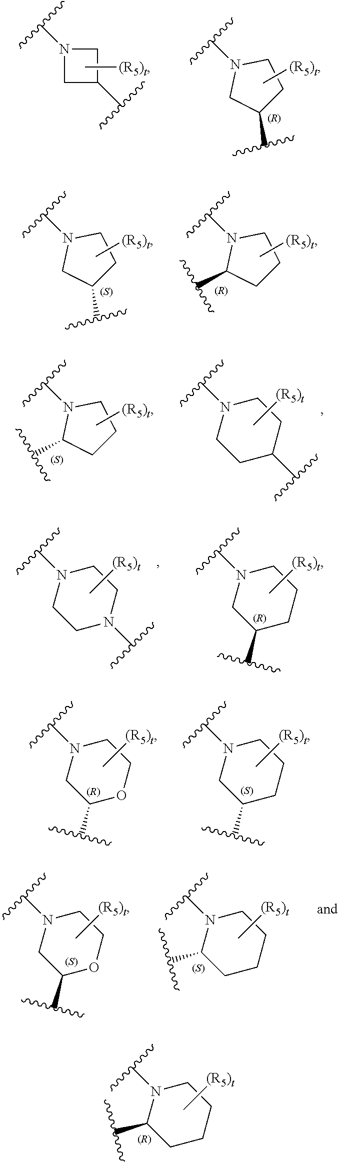
with R₅ being C₁₋₃-alkyl, F, —CH₂CN or —CN and t being 0, 1 or 2, particularly 0.

11. The method according to claim 1, wherein the first and second scaffold are selected from a moiety of formula 3a, 3b, or 3c, particularly 3a, 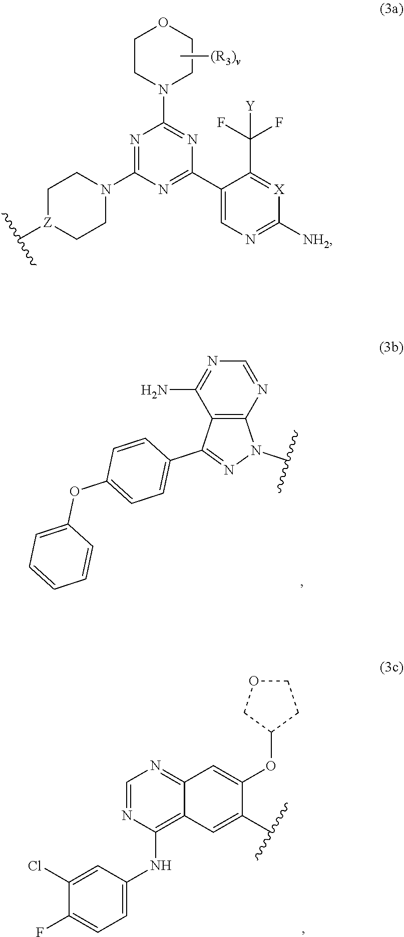
wherein
X is CH or N,
Y is H or F,
Z is O or N, particularly N,
R₃ is C₁₋₃-alkyl or two residues R₃ form a bridge —(CH₂)ᵣ— with r being 1, 2 or 3, particularly R₃ is C₁₋₃-alkyl, more particularly CH₃,
v is 0, 1, 2, 3 or 4, particularly 0, 1 or 2, more particularly 0 or 1, even more particularly 0.

12. The method according to claim 1, wherein the target protein is an enzyme that comprises a cysteine in proximity to a binding site of a reversibly binding inhibitor.

13. The method according to claim 1, wherein the target protein is a protein kinase, particularly a tyrosine kinase.

14. The method according to claim 1, wherein the target protein is selected from PI3K (phosphoinositide 3-kinase), BTK (Bruton's tyrosine kinase) EGFR (epidermal growth factor receptor).

15. The method according to claim 1, wherein the target protein is PI3K (phosphoinositide 3-kinase), particularly PI3Kα.
